(19) United States
(12) Patent Application Publication
(10) Pub. No.: US 2015/0329865 A1
(43) Pub. Date: Nov. 19, 2015

(54) COMPOSITION FOR TREATING EPSTEIN-BARR VIRUS INFECTION, COMPRISING EPSTEIN-BARR VIRUS MICRO RNA INHIBITOR

(71) Applicant: The Catholic University of Korea Industry-Academic Cooperation Foundation, Seoul (KR)

(72) Inventors: Suk Kyeong LEE, Seoul (KR); Yu-Jin Jung, Seoul (KR); Hoyun Choi, Incheon (KR); Hyoji Kim, Bucheon-si (KR)

(21) Appl. No.: 14/702,463

(22) Filed: May 1, 2015

(30) Foreign Application Priority Data
May 16, 2014 (KR) 10-2014-0058765

(51) Int. Cl. C12N 15/113; C12N 7/00; C12Q 1/70

(52) U.S. Cl. CPC C12N 15/1133; C12Q 1/705; C12N 7/00; C12N 2710/16263; C12Q 2600/136; C12Q 2600/158

(57) ABSTRACT

Provided is a composition comprising an Epstein-Barr virus microRNA inhibitor for treating Epstein-Barr virus infection, and a method using Epstein-Barr virus microRNA for screening a therapeutic agent for treating Epstein-Barr virus infection. The provided composition enables one to induce the lytic cycle of EBV such that EBV-infected cells are destroyed by a host immune system. Therefore, the composition can be effectively used for the prevention or treatment of diseases, including various cancers, caused by EBV infection. Moreover, the provided method enables one to screen a therapeutic agent having excellent antiviral effect for treating Epstein-Barr virus infection by inducing Epstein-Barr virus lytic cycle.

### CROSS-REFERENCE TO RELATED PATENT APPLICATION

This application claims priority under 35 U.S.C. §119(a) to Korean Patent Application No. 10-2014-0058765 to Suk Kyeong LEE, Yu-Jin JUNG, Hoyun CHOI and Hyoji KIM, entitled “COMPOSITION FOR TREATING EPSTEIN-BARR VIRUS INFECTION, COMPRISING EPSTEIN-BARR VIRUS MICRO RNA INHIBITOR,” filed May 16, 2014, the disclosure of which is incorporated by reference in its entirety.

### INCORPORATION BY REFERENCE OF SEQUENCE LISTING PROVIDED ELECTRONICALLY

An electronic version of the Sequence Listing is filed herewith, the contents of which are incorporated by reference in their entirety. The electronic file was created on Apr. 24, 2015, is 10 kilobytes in size, and titled 436SEQ001.txt. A substitute Sequence Listing is filed electronically herewith, the contents of which are incorporated by reference in their entirety. The electronic file was created on May 28, 2015, is 10 kilobytes in size, and titled 436SEQ002.txt.

### BACKGROUND OF THE INVENTION

1. Field of the Invention

The present invention relates to a composition for treating Epstein-Barr virus infection, comprising an Epstein-Barr virus microRNA inhibitor, and a method for screening a therapeutic agent for treating Epstein-Barr virus infection using Epstein-Barr virus microRNA.

This study was supported by Gyeonggi-do Regional Research Center (GRRC).

2. Description of the Related Art

Epstein-Barr virus (hereinafter referred to as EBV) is a human gamma-herpesvirus and is known to cause granular fever and hairy leukoplakia in AIDS patients and to trigger autoimmune diseases such as polymyositis, dermatomyositis, systemic lupus erythematosus, rheumatic arthritis, etc. Moreover, EBV is known to cause various malignant tumors including Burkitt's lymphoma, Hodgkin's disease, nasopharyngeal cancer, nasal natural killer/T-cell lymphoma, and gastric cancer.

The life cycle of all herpesviruses, including EBV, can be divided into latent and lytic cycles. During the latent cycle, EBV maintains latency during which it tethers its genome to the DNA of a host cell to express only a few proteins and is replicated along with host cell proliferation. On the contrary, during the lytic cycle, the virus undergoes lytic replication during which it expresses a number of viral genes to produce progeny viruses and then lyses the host cell to release progeny viruses.

Virus is generally known to go through the lytic cycle when the host environment is not good. When going through the lytic cycle, the virus may have higher toxicity to the host than during the latent cycle. However, the virus in the latent cycle evades host immune surveillance and continuously causes disease to the host, while the virus in the lytic cycle is exposed to host immune system and thus is vulnerable to destruction. Therefore, the induction of the viral lytic cycle has been actively studied as a target of antiviral agents for treating various diseases including tumors caused by latent infection of the virus.

### SUMMARY OF THE INVENTION

The present invention aims at providing a composition for preventing or treating Epstein-Barr virus infection, comprising a miR-BART20-5p inhibitor. Moreover, the present invention aims at providing a composition for inducing Epstein-Barr virus lytic cycle, comprising a miR-BART20-5p inhibitor. Furthermore, the present invention aims at providing a method for promoting the induction of Epstein-Barr virus lytic cycle ex vivo or in vitro using a miR-BART20-5p inhibitor. In addition, the present invention aims at providing a method for inhibiting the induction of Epstein-Barr virus lytic cycle ex vivo or in vitro using miR-BART20-5p. Also, the present invention aims at providing a method for screening a therapeutic agent for treating Epstein-Barr virus infection using miR-BART20-5p.

To achieve the above objects, the present invention provides a composition for inducing Epstein-Barr virus lytic cycle, comprising a miR-BART20-5p inhibitor.

The present invention provides a method for inducing Epstein-Barr virus lytic cycle in a subject, comprising administering miR-BART20-5p to a subject in need thereof.

The present invention provides a composition for preventing or treating Epstein-Barr virus infection, comprising a miR-BART20-5p inhibitor as an active ingredient.

The present invention provides a method for treating Epstein-Barr virus infection of a subject, comprising administering miR-BART20-5p inhibitor to a subject in need thereof.

Epstein-Barr virus infection may be selected from the group consisting of granular fever, hairy leukoplakia, polymyositis, dermatomyositis, systemic lupus erythematosus, rheumatic arthritis, and cancer. Preferably, the Epstein-Barr virus infection may be cancer. The cancer may be selected from the group consisting of Epstein-Barr virus-positive Burkitt's lymphoma, Hodgkin's lymphoma, nasopharyngeal cancer, nasal natural killer/T-cell lymphoma, and gastric cancer. More preferably, the cancer may be Epstein-Barr virus-positive gastric cancer. However, the type of the disease is not limited thereto, and all diseases triggered by the Epstein-Barr virus infection are included.

The miR-BART20-5p inhibitor may be a nucleotide that complementarily binds to miR-BART20-5p to inhibit the activity of miR-BART20-5p. In an embodiment of the present invention, the miR-BART20-5p inhibitor is a sequence comprising 5′-nnnnnnnnnnnnnGCCUGCUn-3′ (SEQ ID NO: 1) and comprising a total of 21 to 25 nucleotides and have miR-BART20-5p inhibitory activity. The region “GCCUGCU” in SEQ ID NO: 1 is a sequence complementary to the core sequence that binds to BRLF1 3′UTR and BZLF1 3′UTR in miR-BART20-5p to exhibit the activity of miR-BART20-5p. Moreover, the miR-BART20-5p inhibitor may be 5′-nnAnUGAAnnCAnGCCUGCUn-3′ (SEQ ID NO: 2). In SEQ ID NOs: 1 and 2, n may be any of A, G, T, U and C. In the present invention, U and C are used interchangeably. Preferably, the miR-BART20-5p inhibitor may be a miR-BART20-5p antisense RNA represented by SEQ ID NO: 3. However, the type of the miR-BART20-5p inhibitor is not limited thereto. Various modifications to increase affinity and stability may be added to the miR-BART20-5p inhibitor of the present invention. For example, sugar modifications such as locked nucleic acid, 2′fluroro, 2′-O-methoxyethyl, 2′-O-methyl, etc. or backbone modifications such as morpholino linkage, phosphorothioate linkage, etc. may be added. Preferably, the locked nucleic acid modification may be achieved by the miR-BART20-5p inhibitor. However, these nucleic acid modifications are well known in the art (for example, see Inhibition of microRNA function by antimiR oligonucleotides, Jan Stenvang et al., Silence 2012, 3:1, FIG. 2) and may be easily carried out by those skilled in the art.

In the present invention, the effect of the miR-BART20-5p inhibitor on the prevention or treatment of Epstein-Barr virus infection is achieved by increasing the expression of BRLF1 and BZLF1.

EBV produces a total of 44 miRNAs, which are generated by BamHI fragment H rightward open reading frame 1 (BHRF1) or BamHI A rightward transcripts (BARTs). One of them is miR-BART20-5p that binds to the 3′ untranslated region (3′UTR) of immediate early genes, BRLF1 and BZLF1, which play a key role in the induction of EBV lytic cycle, to inhibit the expression of BRLF1 and BZLF1, rendering EBV to maintain the latent cycle.

It is known that EBV very effectively evades host immunity during the latent cycle, but during the lytic cycle, it expresses proteins acting as potent immunogens and thus is highly likely to be removed by host immune system. Whether EBV goes through the latent cycle or the lytic cycle is determined by the expression of certain lytic genes, and it is known that the expression of immediate early genes, BRLF1 and BZLF1, among the lytic genes, plays a key role in the induction of EBV lytic cycle.

There are several genes involved in the lytic cycle of EBV, which are shown in the following Table 1:

TABLE 1

| | Expression time | Gene name |
|---|---|---|
| EBV lytic | Immediate early genes | BRLF1, BZLF1 |
| genes | Early genes | BMRF1, BALF5, BHRF1 |
| | Late genes | gp350/220, gp35 |

In the above Table 1, the earliest expressed genes (immediate early genes) that induce the lytic cycle of EBV include BRLF1 and BZLF1. Upon the expression of BRLF1 and BZLF1, the expression of BRLF1 and BZLF1 is facilitated by positive feedback. Subsequently, the expression of early genes such as BMRF1, BALF5 and BHRF1 is induced, the expression of late genes such as gp350/220 and gp35 is then induced, and finally lytic replication of EBV occurs.

Therefore, the composition comprising the miR-BART20-5p inhibitor as an active ingredient of the present invention inhibits the activity of miR-BART20-5p to promote the expression of BRLF1 and BZLF1, which induces the lytic cycle of EBV such that EBV is exposed to host immune system and thus is vulnerable to destruction, thus preventing or treating EBV infection.

The pharmaceutical composition of the present invention may be prepared using pharmaceutically suitable and biologically acceptable adjuvants other than the active ingredient, and examples of the adjuvants may include excipients, disintegrants, sweeteners, binders, coating agents, extenders, lubricants, glidants, and flavoring agents.

The composition according to the present invention may comprise at least one pharmaceutically acceptable carrier for administration other than the above-described active ingredient and may be formulated into a pharmaceutical composition.

The pharmaceutical composition of the present invention may be formulated into dosage forms such as solutions, suspensions, emulsions, drops, dispersions, powders, syrups, juices, granules, tablets, coated tablets, capsules, suppositories, creams, lotions, ointments, gels, patches, sprays, sticking plasters, injectable solutions, active compound sustained-release agents, etc.

The active ingredient in the composition of the present invention may preferably be contained in an amount of 0.00001 to 10 wt % with respect to the total weight of the composition, but not limited thereto.

The composition or the active ingredient of the present invention may be administered in a typical manner via any route such as intravenous, intraarterial, intraperitoneal, intramuscular, intrasternal, percutaneous, subcutaneous, intranasal, inhalation, topical, rectal, oral, intraocular or intradermal route, preferably via intravenous, subcutaneous, or percutaneous route, but not limited thereto.

In the composition of the present invention, the dosage of the active ingredient may be regulated in the range of 0.00001 mg/kg˜50 mg/kg based on a 60 kg adult. However, an optimal dosage may be easily determined by those skilled in the art and may be adjusted depending on various factors including the type of disease, the severity of disease, the content of the active ingredient and other ingredients contained in the composition, the type of dosage form, a patient's age, weight, general health status, sex, and diet, administration time, administration route, secretion rate of the composition, treatment period, co-administered drugs, etc.

Moreover, the present invention provides a method for preventing or treating Epstein-Barr virus infection by administering a therapeutically effective amount of the composition or active ingredient of the present invention.

As used herein, the term “therapeutically effective amount refers to the amount of the active ingredient or pharmaceutical composition that will elicit the biological or medical response of an animal or human that is being sought by a researcher, veterinarian, medical doctor or other clinician and includes an amount that induces alleviation of a disease or disorder to be treated. It will be apparent to those skilled in the art that the therapeutically effective amount and the administration times of the active ingredient of the present invention will vary depending on the desired effect.

The present invention provides a method for inhibiting the induction of Epstein-Barr virus lytic cycle ex vivo or in vitro, the method comprising the step of inhibiting the induction of Epstein-Barr virus lytic cycle by treating Epstein-Barr virus-positive cells with miR-BART20-5p represented by SEQ ID NO: 4 or a miR-BART20-5p analogue represented by SEQ ID NO: 5 or SEQ ID NO: 6.

In the present invention, the “miR-BART20-5p” includes those produced by EBV and those artificially synthesized and is represented by SEQ ID NO: 4. Artificially-synthesized miR-BART20-5p that has a sequence completely identical to that produced by EBV refers to a “miR-BART20-5p mimic”. Moreover, sequences with an activity of inhibiting the expression of BRLF1 and BZLF1 and has a sequence of 5′-nAGCAGGCnUGnnUUCAnUnn-3′ (SEQ ID NO: 5) or 5′-nAGCAGGCnnnnnnnnnnnnn-3′ (SEQ ID NO: 6) is referred to as a “miR-BART20-5p analogue”. In SEQ ID NOs: 5 and 6, n may be any of A, G, T and U. In the present invention, U and C are used interchangeably. 5′-AGCAGGC-3′ of SEQ ID NO: 5 is a sequence located at the 5′ end of miR-BART20-5p and is the core sequence that binds to BRLF1 3′UTR and BZLF1 3′UTR to exhibit the activity of miR-BART20-5p. Therefore, the sequence represented by SEQ ID NO: 6, including this sequence, can be used as miR-BART20-5p in the present invention.

In the present invention, the term “Epstein-Barr virus-positive cells” refers to Epstein-Barr virus-infected cells.

Moreover, the present invention provides a method for screening a therapeutic agent for treating Epstein-Barr virus infection, the method comprising the steps of:

1) treating Epstein-Barr virus-positive cells with miR-BART20-5p represented by SEQ ID NO: 4 or a miR-BART20-5p analogue represented by SEQ ID NO: 5 or SEQ ID NO: 6;

2) treating the cells with a candidate for therapeutic agents; and

3) determining the induction of lytic cycle in the cells.

In step 1) of the above method, the Epstein-Barr virus-positive cells are Epstein-Barr virus-infected cells.

In the above method, step 3) may be carried out by determining the expression level of Epstein-Barr virus genes. Since Epstein-Barr virus rarely expresses proteins in the latent cycle, it may be confirmed whether the lytic cycle is induced by the candidate that directly inhibits miR-BART20-5p or offsets or overcomes the effect of miR-BART20-5p when the expression of Epstein-Barr virus genes is higher than that in the latent cycle which is determined using any method such as Western blot, RT-PCR, etc.

Moreover, step 3 of the above method may be carried out by determining the expression of BRLF1 and BZLF1 of Epstein-Barr virus.

The present invention provides a method for screening a therapeutic agent for treating Epstein-Barr virus infection, the method comprising the steps of:

1) treating cells that express BRLF1 3′UTR or BZLF1 3′UTR of Epstein-Barr virus with miR-BART20-5p represented by SEQ ID NO: 4 or a miR-BART20-5p analogue represented by SEQ ID NO: 5 or SEQ ID NO: 6;

2) treating the cells with a candidate for therapeutic agents; and

3) determining the expression of BRLF1 3′UTR or BZLF1 3′UTR in the cells.

In step 1), the cells that express the BRLF1 3′UTR or BZLF1 3′UTR of Epstein-Barr virus may be a cell line that carries a vector that in which a reporter gene is inserted in the BZLF1 3′UTR and/or BRLF1 3′UTR. The BRLF1 3′UTR may be a sequence represented by SEQ ID NO: 7, and BZLF1 3′UTR may be a sequence represented by SEQ ID NO: 8. However, a cell line that carries a vector that contains only a sequence that includes at least one copy of 5′-GCUCGA-3′, which corresponds to a part of SEQ ID NO: 7 or SEQ ID NO: 8, not to the whole thereof, fused to the reporter gene may also be used in the present invention.

In step 3), the expression of BRLF1 3′UTR or BZLF1 3′UTR may be determined by determining the expression of the reporter gene that is expressed together with the BRLF1 3′UTR or BZLF1 3′UTR. The reporter gene may include various luciferases such as Renilla luciferase, various fluorescent protein genes such as GFP, RFP, etc., ferritin genes, transferrin receptor genes, etc., but not limited thereto.

Moreover, the present invention provides a method for inducing Epstein-Barr virus lytic cycle ex vivo or in vitro, the method comprising the step of inducing Epstein-Barr virus lytic cycle by treating Epstein-Barr virus-positive cells with a miR-BART20-5p inhibitor represented by SEQ ID NO: 1, SEQ ID NO: 2 or SEQ ID NO: 3.

### Advantageous Effects

As described above, the use of the composition comprising the miR-BART20-5p inhibitor as an active ingredient of the present invention enables to induce the lytic cycle of EBV such that EBV-infected cells are destroyed by host immune system. Therefore, the composition of the present invention can be effectively used for the prevention or treatment of diseases, including various cancers, caused by EBV infection. Moreover, the use of the method of the present invention enables to screen a therapeutic agent having excellent antiviral effect for treating Epstein-Barr virus infection by inducing Epstein-Barr virus lytic cycle.

### BRIEF DESCRIPTION OF THE DRAWINGS

The above and other features and advantages of the present invention will become more apparent by describing in detail exemplary embodiments thereof with reference to the attached drawings in which:

FIG. 1 shows the effect of a miR-BART20-5p mimic on the inhibition of BRLF1 expression in AGS cell line, determined by luciferase reporter assay;

FIG. 2 shows the effect of a miR-BART20-5p mimic and a mutant (miR-BART20-5pm) of the miR-BART20-5p mimic on the inhibition of BRLF1 and BZLF1 expression in HEK293T cell line, determined by luciferase reporter assay;

FIG. 3 shows the effect of a miR-BART20-5p mimic on the inhibition of BRLF1 and BZLF1 expression after mutation of target sites in the 3′UTRs of BRLF1 and BZLF1 such that miRNAs do not bind thereto in HEK293T cell line, determined by luciferase reporter assay;

FIG. 4 shows Target sites 1 and 2 expected to have seed match sites to a miR-BART20-5p mimic in the 3′UTRs of BRLF1 and BZLF1 and the regions where mutations are introduced;

FIGS. 5A and 5B are schematic diagrams showing the regions where mutations are introduced in FIG. 4;

FIG. 6A shows the expression of lytic genes, BRLF1 and BZLF1 by TPA treatment, and FIG. 6B shows the reduction in BRLF1 and BZLF1 mRNA levels by treatment of a miR-BART20-5p mimic despite TPA treatment;

FIG. 7 shows the decreased expression of lytic genes, BRLF1, BZLF1, BMRF1, BALF5, and BHRF1 by treatment of a miR-BART20-5p mimic, determined by Western blot;

FIG. 8 is a graph showing the results of FIG. 7;

FIG. 9 shows the increased expression of EBV miR-BART20-5p mRNAs in EBV by TPA treatment;

FIG. 10 shows the significantly reduced expression of EBV miR-BART20-5p mRNA by treatment with a miR-BART20-5p inhibitor (LNA-miR-BART20-5pi);

FIG. 11 shows the increased expression of BRLF1 and BZLF1 mRNAs by treatment with a miR-BART20-5p inhibitor in AGS-EBV cell line;

FIG. 12 shows the increased expression of BRLF1 and BZLF1 mRNAs by treatment with a miR-BART20-5p inhibitor in SNU-719 cell line and YCCEL1 cell line;

FIG. 13 shows the increased expression of BRLF1, BZLF1, and BALF5 proteins by treatment with a miR-BART20-5p inhibitor, determined by Western blot;

FIG. 14 shows that siBRLF1, siBZLF1 and miR-BART20-5p mimic cause the reduction of BRLF1 and BZLF1 expression, determined by Western blot;

FIG. 15 shows that siBRLF1, siBZLF1 and miR-BART20-5p mimic cause the reduction in the production of progeny viruses; and

FIG. 16 shows that a miR-BART20-5p inhibitor causes the increase in the production of progeny viruses.

### DETAILED DESCRIPTION OF THE INVENTION

The reagents and solvents mentioned below were purchased from Sigma Aldrich® unless otherwise stated.

Hereinafter, preferred Examples are provided for better understanding of the present invention. However, the following Examples are provided to facilitate understanding of the present invention, and the scope of the present invention is not limited by the following Examples.

### Preparation Example 1

### Preparation of miR-BART20-5p Mimic and Antisense RNA

A miR-BART20-5p mimic of EBV was purchased from Genolution Pharmaceuticals (Seoul, South Korea). Moreover, an antisense RNA against miR-BART-20-5p was purchased from Exiqon (California, USA) (hereinafter referred to as “LNA-miR-BART20-5pi” in the following Examples and drawings). The sequences thereof are shown in the following Table 2:

TABLE 2

| | | SEQ ID |
|---|---|---|
| Name | Sequence | NO |
| LNA-miR- | 5′ GGAAUGAAGACAUGCCUGCUA 3′ | 3 |
| BART-20- | | |
| 5pi | | |
| miR- | 5′ UAGCAGGCAUGUCUUCAUUCC 3′ | 4 |
| BART20- | | |
| 5p mimic | | |

### Example 1

### Determination of Effect of miR-BART20-5p on the Inhibition of BRLF1 and BZLF1 Expression and Determination of Seed Match Sites in miR-BART20-5p

1-1. Preparation of Cell Lines

AGS cell line, an EBV-negative gastric cancer cell line, and HEK293T cell line, a human embryonic kidney cell line, were prepared. The AGS cell line was cultured in RPMI 1640 (Gibco) medium containing 10% fetal bovine serum (FBS). Moreover, the HEK293T cell line was cultured in Dulbecco's Modified Eagle's Medium (DMEM) supplemented with 10% FBS. Furthermore, AGS-EBV cell line, an AGS cell line infected with EBV, was cultured in RPMI 1640 (Gibco) medium containing 10% FBS and G418.

1-2. Construction of Plasmid Containing BRLF1 3′UTR and Plasmid Containing BZLF1 3′UTR

The full-length sequences corresponding to the BRLF1 3′UTR (SEQ ID NO: 7) and the BZLF1 3′UTR (SEQ ID NO: 8) of EBV were amplified from the cDNA of AGS-EBV cells prepared in Example 1-1 using a pair of primers of SEQ NOs: 9 and 10 and a pair of primers of SEQ ID NOs: 11 and 12 shown in the following Table 3, respectively. The amplified BRLF1 3′UTR and BZLF1 3′UTR were then cloned into Xho I/Not I sites between the Renilla luciferase coding sequence and the poly(A) tail of the psiCHECK-2 plasmid (Promega, Madison, Wis.). The constructed plasmids were named a psiC-BZLF1 plasmid and a psiC-BRLF1 plasmid, respectively.

TABLE 3

| | | | SEQ |
|---|---|---|---|
| Gene | | | ID |
| name | Direction | Sequence | NO |
| BRLF1 | Forward | 5′-TCGACTCGAGGAGCCACAGGCATTGC | 9 |
| | | TAA-3′ | |
| | Reverse | 5′-GGCCGCGGCCGCCAAAGAGAGCCGAC | 10 |
| | | AGGAAG-3′ | |
| BZLF1 | Forward | 5′- TCGACTCGAGCGAGGATCTCTTAAA | 11 |
| | | TTTCTAACTCC-3′ | |
| | Reverse | 5′-GGCCGCGGCCGCCAAAGAGAGCCGAC | 12 |
| | | AGGAAG-3′ | |

1-3. Luciferase Reporter Assay on Plasmid Containing BRLF1 3′UTR in AGS Cell Line

AGS cell line was cultured in the same manner as in Example 1-1.

The cultured AGS cell line was co-transfected with the miR-BART20-5p mimic prepared in Preparation Example 1 and the psiC-BRLF1 plasmid prepared in Example 1-2. Scrambled control (5′-CCUCGUGCCGUUCCAUCAGGUAGUU-3′, SEQ ID NO: 13, purchased from Genolution Pharmaceuticals (Seoul, South Korea), all scrambled controls used below are the same) was used as control for the miR-BART20-5p mimic. Luciferase activity was measured 48 hours post-transfection using the Dual-Glo luciferase reporter assay system (Promega).

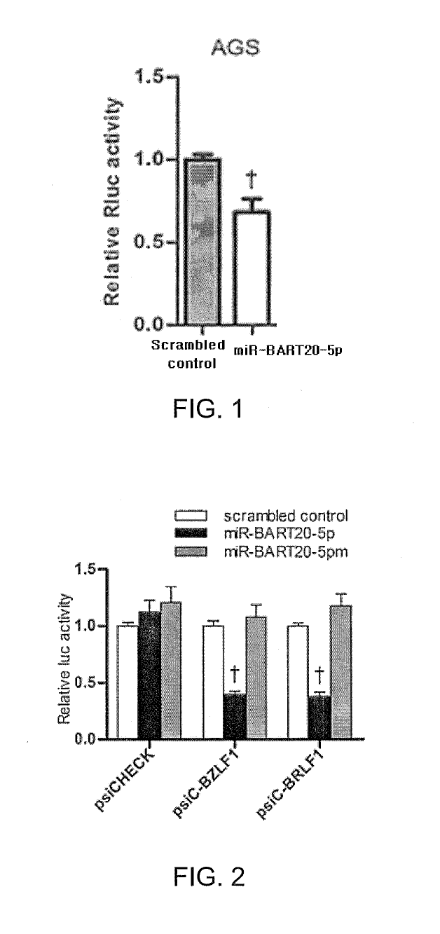

The results are shown in FIG. 1. It can be seen from FIG. 1 that the miR-BART20-5p mimic reduced the luciferase activity in the AGS cell line more than 30% compared to the scrambled control. This confirms that the miR-BART20-5p mimic binds to the 3′UTR of BRLF1 to inhibit the expression of BRLF1.

1-4. Construction of Mutant of miR-BART20-5p Mimic

It was expected that the mutation of nucleotides 4-6 among nucleotides 2-8 at the 5′ end of miR-BART20-5p, which have seed match sites to the 3′UTR of BRLF1 and the 3′UTR of BZLF1, makes the miR-BART20-5p inactive. Therefore, a mutant RNA was constructed by mutating these sequences from 5′-CAG-3′ to 5′-GUC-3′ (bolded and underlined in the following table) and named miR-BART20-5pm (SEQ ID NO: 14). The sequence of miR-BART20-5pm is shown in the following Table 4:

TABLE 4

| | | SEQ ID |
|---|---|---|
| Name | Sequence | NO |
| miR-BART20- | 5′ UAGGUCGCAUGUCUUCAUUCC 3′ | 14 |
| 5pm | | |

1-5. Luciferase Reporter Assay in HEK293T Cell Line

HEK293T cell line was seeded in a 96-well plate at 3×10³ cells/well and cultured at a 37° C. incubator for 24 hours.

The cultured HEK293T cell line was treated with either the miR-BART20-5p mimic prepared in Preparation Example 1 or the miR-BART20-5pm prepared in Example 1-3, transfected with the psiC-BRLF1 plasmid or the psiC-BZLF1 plasmid prepared in Example 1-2, respectively, and then cultured at a 37° C. incubator. The scrambled control was used as control for the miR-BART20-5p mimic.

Luciferase activity was measured 48 hours post-transfection using the Dual-Glo luciferase reporter assay system (Promega).

The results are shown in FIG. 2. It can be seen from FIG. 2 that when the HEK293T cell line was treated with the miR-BART20-5p mimic and transfected with the psiC-BRLF1 plasmid or the psiC-BZLF1 plasmid, respectively, the miR-BART20-5p mimic reduced the luciferase activity more than 60% compared to the control. On the contrary, both the control and the HEK293T cell line treated with the miR-BART20-5pm and transfected with the psiC-BRLF1 plasmid or the psiC-BZLF1 plasmid, respectively, exhibited no reduction in luciferase activity.

This confirms that the miR-BART20-5p binds to the 3′UTRs of BRLF1 and BZLF1 to inhibit the expression of BRLF1 and BZLF1, and since this phenomenon occurs sequence-specifically, miR-BART20-5p loses its ability even when the seed sequences are partially mutated.

### Example 2

### Determination of Seed Match Sites Between miR-BART20-5p and BRLF1 and BZLF1

2-1. Construction of Plasmids Containing Mutant BRLF1 3′UTR and Mutant BZLF1 3′UTR, Respectively

The following experiment was carried out to determine the sites of the 3′UTRs of BZLF1 and BRLF1 to which miR-BART20-5p complementarily binds.

BZLF1 and BRLF1 are bicistronic genes, and the full-length sequence of BZLF1 is encoded inside the 3′UTR of BRLF1. Therefore, the BZLF1 3′UTR is contained in the BRLF1 3′UTR.

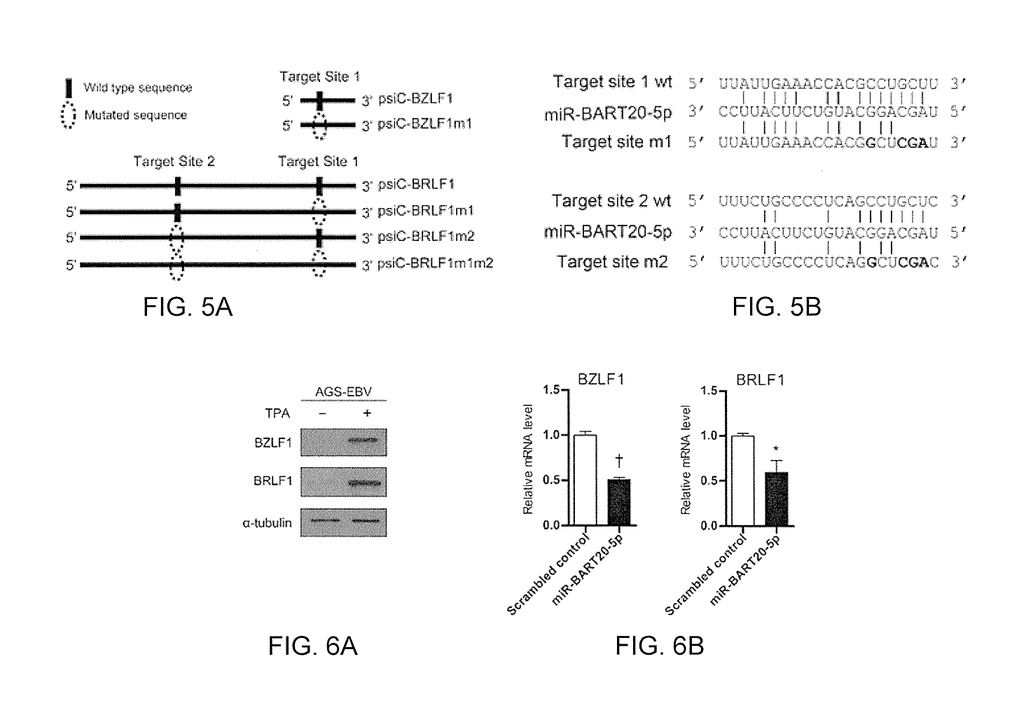

From the study of the 3′UTRs of BZLF1 and BRLF1, it was expected that the 5′-CCUGCU-3′ (shown in red and double underlined in the highlighted portions) located on Target site 1 (highlighted in green and single underlined in FIG. 4. commonly present in 3′UTRs of BZLF1 and BRLF1) and Target site 2 (highlighted in yellow and single overlined in the 3′UTR of BRLF1 in FIG. 4) was the front part of seed match sites to which miR-BART20-5p binds. FIGS. 5A and 5B are schematic diagrams showing the regions where mutations were introduced in FIG. 4.

Accordingly, the region corresponding to 5′-CCUGCU-3′ in Target site 1 was mutated to 5′-GCUCGA-3′ to produce a mutant of BZLF1 3′UTR (SEQ ID NO: 15, hereinafter referred to as BZLF1m1) and a mutant of BRLF1 3′UTR (SEQ ID NO: 16, hereinafter referred to as BRLF1m1) using an EZchange™ site-directed mutagenesis kit (Enzynomics, Daejeon, South Korea).

Moreover, the region corresponding to 5′-CCUGCU-3′ in Target site 2 was mutated to 5′-GCUCGA-3′ to produce a mutant of BRLF1 3′UTR (SEQ ID NO: 17, hereinafter referred to as BRLF1m2) using an EZchange™ site-directed mutagenesis kit (Enzynomics, Daejeon, South Korea).

Furthermore, in the BRLF1 3′UTR, the regions corresponding to 5′-CCUGCU-3′ in Target site 1 and Target site 2 were all mutated to 5′-GCUCGA-3′ to produce a mutant of BRLF1 3′UTR (SEQ ID NO: 18, hereinafter referred to as BRLF1m1m2) using an EZchange™ site-directed mutagenesis kit (Enzynomics, Daejeon, South Korea).

The respective mutant genes were amplified using a pair of primers of SEQ ID NOs: 19 and 20, a pair of primers of SEQ ID NOs: 21 and 22, and psiC-BRLF1 and psiC-BZLF1 plasmids as templates. The constructed plasmids were named a psiC-BRLF1m1 plasmid, a psiC-BRLF1m2 plasmid, a psiC-BRLF1m1m2 plasmid, and a psiC-BZLF1m1 plasmid, respectively.

The primers used for the amplification of the respective genes are shown in the following Table 5:

TABLE 5

| | | | SEQ |
|---|---|---|---|
| Gene Name | Direction | Sequence | ID NO |
| BRLF1m2 | Forward | 5′-CTGAGAATGCTTATCAAGCT | 19 |
| | | TATGCAGCAC-3′ | |
| | Reverse | 5′-GTCGAGCCTGAGGGGCAGGA | 20 |
| | | AACCACG-3′ | |
| BRLF1m1 | Forward | 5′-CACGCCTCGTTTACTAATGG | 21 |
| (using | | AATATTAATAAATAT-3′ | |
| primer | Reverse | 5′-ATCGAGCCGTGGTTTCAATA | 22 |
| common to | | ACG-3′ | |
| BZLF1m1 | | | |
| and | | | |
| BRLF1m1m2) | | | |

2-2. Luciferase Reporter Assay on Plasmid Containing Mutant BRLF1 3′UTR and Plasmid Containing Mutant BZLF1 3′UTR

It was examined whether the luciferase activity was reduced when the HEK293T cell line was co-transfected with either the miR-BART20-5p mimic or miR-BART20-5pm and the psiC-BRLF1m1, psiC-BRLF1m2, psiC-BRLF1m1m2, or psiC-BZLF1m1 plasmid prepared in Example 2-1. A psiCHECK-2 luciferase reporter vector without inserted gene was used as control for the plasmid, and the scrambled control was used as control for the miR-BART20-5p mimic.

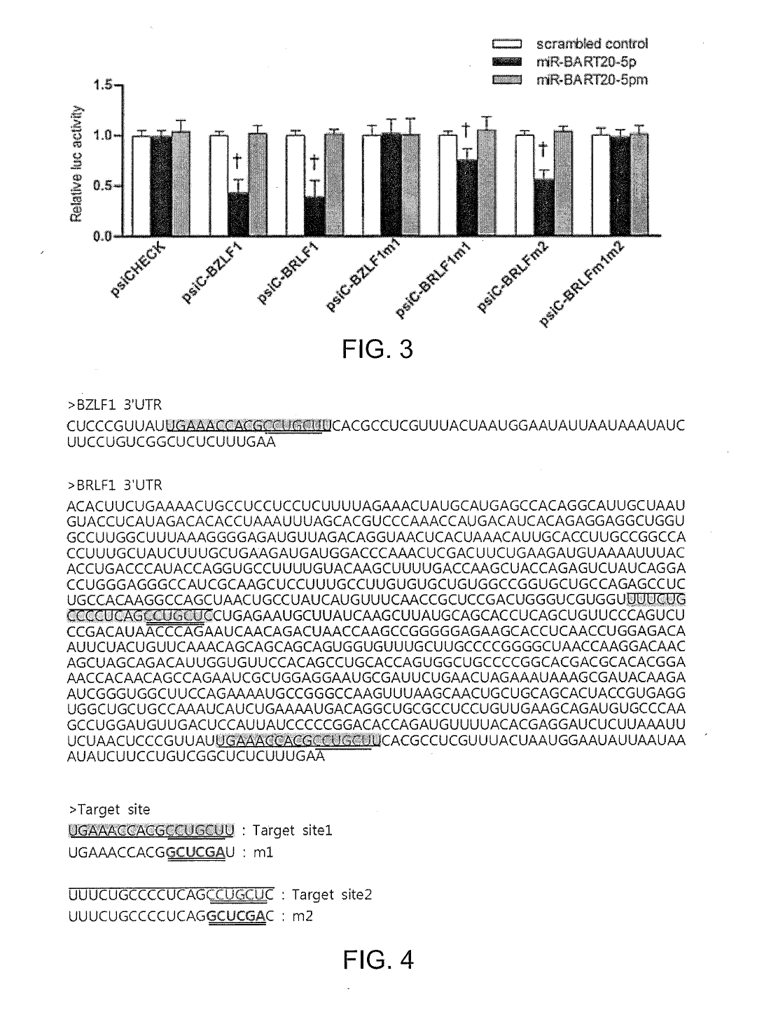

The results are shown in FIG. 3. Plasmids with inserted mutant genes are shown in the fourth to seventh groups of bars from the left of FIG. 3.

It can be seen from FIG. 3 that the miR-BART20-5p mimic does not reduce the luciferase activity of the psiC-BZLF1m1 plasmid. The miR-BART20-5p mimic partially reduced the luciferase activity of the psiC-BRLF1m1 and psiC-BRLF1m2 plasmids, but the reduction in luciferase activity was much smaller than that of psiC-BRLF1. The miR-BART20-5p mimic did not reduce the luciferase activity of the psiC-BRLF1m1m2 plasmid. This confirms that both sites in BRLF1 expected by the present inventors involve seed match with the miR-BART20-5p mimic.

### Example 3

### Determination of Effect of miR-BART20-5p Mimic on Inhibition of Expression of Lytic Genes of EBV

3-1. Preparation of Cell Line

AGS-EBV cell line, an EBV-positive gastric cancer cell line, was prepared. The AGS-EBV cell line was cultured in RPMI 1640 (Gibco) medium containing 10% fetal bovine serum (FBS) and G418.

3-2. Induction of BRLF1 and BZLF1 Expression by TPA

The AGS-EBV cell line in the latent cycle rarely expresses lytic genes, and thus it is difficult to examine the effect of the miR-BART20-5p mimic. Therefore, the AGS-EBV cell line was treated with 5 nM TPA (12-O-Tetradecanoylphorbol-13-acetate), a well-known lytic cycle inducing agent, for 48 hours to induce the lytic cycle, and the expression of immediate early lytic genes, BRLF1 and BZLF1, was observed by Western blot analysis.

It can be seen from FIG. 6A that the TPA treatment induced the expression of BRLF1 and BZLF1. This confirms that the lytic cycle of EBV was successfully induced by the TPA treatment.

3-3. Determination of Effect of miR-BART20-5p Mimic and TPA Treatment on Expression of Lytic Genes of EBV

The effect of the miR-BART20-5p mimic on the expression of lytic genes of EBV was determined in the following experiment.

(1) Determination of Reduction in BRLF1 and BZLF1 mRNA Levels by Quantitative Real-Time PCR

AGS-EBV cell line was seeded in a 100 mm dish at 1×10⁶ cells/well and then transfected with 30 nM miR-BART20-5p mimic and 30 nM scrambled control, respectively, using Lipofectamine 2000.

After 24 hours, the AGS-EBV cell line was divided into two groups, and only one group was treated with 5 nM TPA for 48 hours. After isolation of RNAs from both groups, cDNA was synthesized using a Mir-X™ First-Strand Synthesis kit, and then the mRNA levels of BRLF1 and BZLF1 were measured by quantitative real-time PCR.

The results are shown in the graph of FIG. 6B. It can be seen from FIG. 6B that the BRLF1 and BZLF1 mRNA levels were reduced by about 50% in the group treated with the miR-BART20-5p mimic compared to the control.

This confirms that the miR-BART20-5p inhibits the expression of lytic genes, BRLF1 and BZLF1 even after treatment of TPA, a lytic replication inducing agent.

(2) Determination of Reduction in Expression of BRLF1, BZLF1, BMRF1, BALF5, and BHRF1 Proteins by Western Blot

AGS-EBV cell line was seeded in a 100 mm dish at 1×10⁶ cells/well and then transfected with 30 nM miR-BART20-5p mimic and 30 nM scrambled control, respectively, using Lipofectamine 2000.

After 24 hours, the AGS-EBV cell line was divided into two groups, and only one group was treated with 5 nM TPA for 48 hours. After isolation of proteins from both groups, the proteins were subjected to electrophoresis on 8% sodium dodecyl sulfate (SDS) gels and transferred to a polyvinylidene fluoride (PVDF) membrane, thus determining the expression level of lytic genes, BRLF1, BZLF1, BMRF1, BALF5, and BHRF1 proteins.

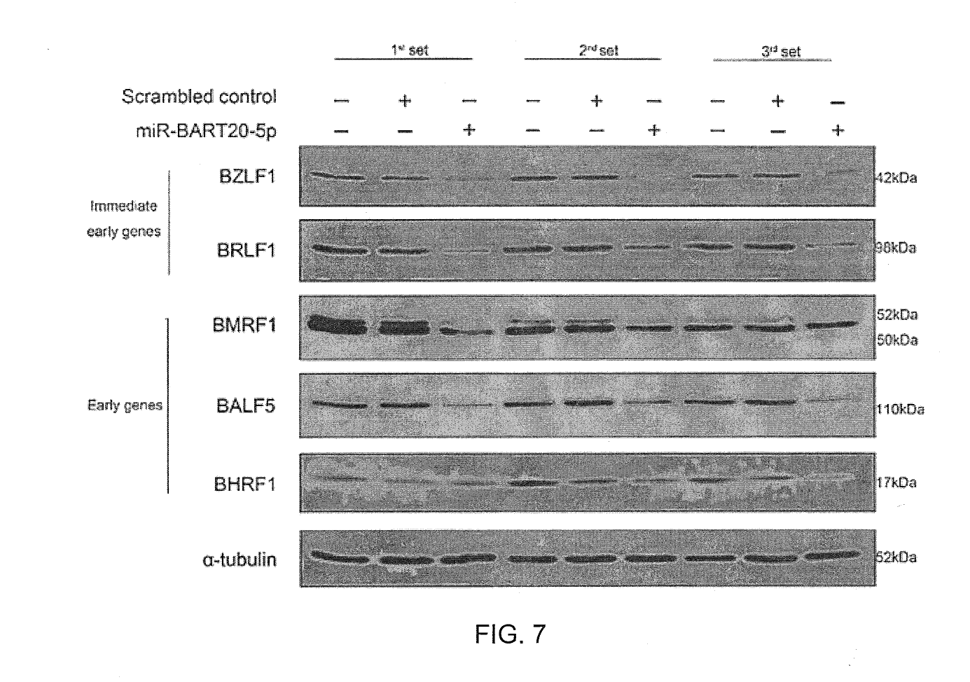

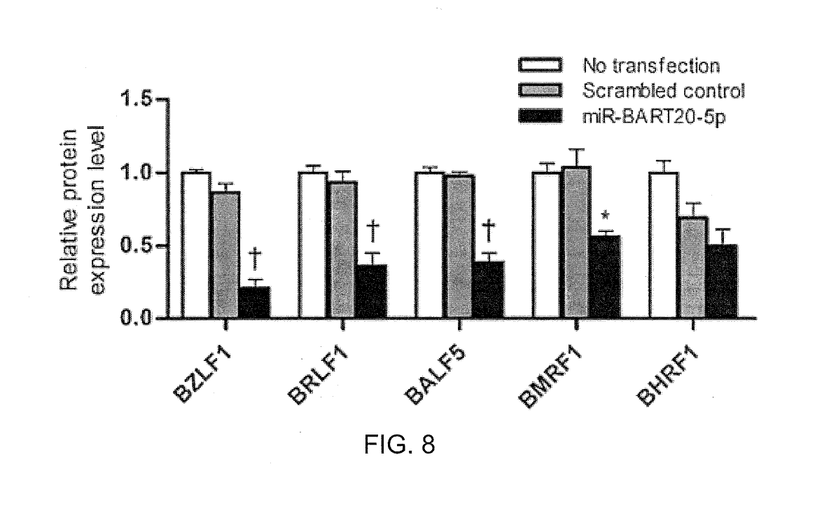

The Western blot results are shown in FIG. 7 and in the graph of FIG. 8. It can be seen from FIGS. 7 and 8, that the expression of BRLF1, BZLF1, BMRF1, BALF5, and BHRF1 proteins was reduced by 50 to 70% in the group treated with the miR-BART20-5p mimic compared to the control.

This confirms that the miR-BART20-5p significantly inhibits the expression of lytic genes, BRLF1, BZLF1, BMRF1, BALF5, and BHRF1 even after treatment of TPA, a lytic replication inducing agent.

### Example 4

### Determination of Change in Expression of miR-BART20-5p in EBV by Induction of Lytic Replication

The AGS-EBV cell line was treated with 5 nM TPA for 48 hours to induce the lytic cycle. After isolation of RNAs, cDNA was synthesized using a Mir-X™ First-Strand Synthesis kit, and then the expression level of miR-BART20-5p was measured by quantitative real-time PCR.

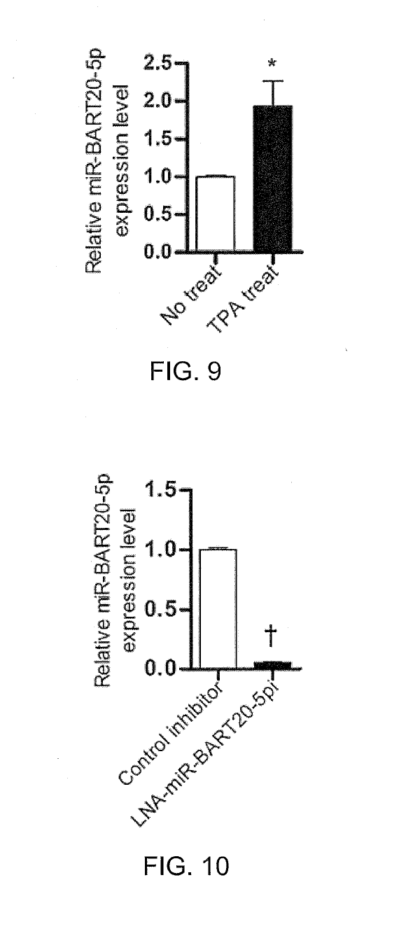

The results are shown in FIG. 9. It can be seen from FIG. 9 that the expression of miR-BART20-5p was increased by the TPA treatment by about 2 times compared to the control without treatment.

### Example 5

### Determination of Effect of SNA-miR-BART20-5pi on Inhibition of Expression of miR-BART20-5p in EBV by Induction of Lytic Replication

AGS-EBV cell line was seeded in a 100 mm dish at 1×10⁶ cells/well and divided into two groups. One group was transfected with 30 nM LNA control (SEQ ID NO: 23, TAACACGTCTATACGCCCA, Exiqon) and the other group was transfected with 30 nM LNA-miR-BART20-5pi using Lipofectamine 2000.

After isolation of RNAs from both groups, cDNA was synthesized using a Mir-X™ First-Strand Synthesis kit, and then the level of miR-BART20-5p was measured by quantitative real-time PCR.

The results are shown in FIG. 10. It can be seen from FIG. 10 that the level of miR-BART20-5p was reduced by more than 98% in the group treated with LNA-miR-BART20-5pi.

### Example 6

### Determination of Effect of LNA-miR-BART20-5pi on Induction of Expression of Lytic Genes in EBV by Induction of Lytic Replication

6-1. Determination of Increase in BRLF1 and BZLF1 mRNA Levels by Quantitative Real-Time PCR

(1) Determination of Increase in BRLF1 and BZLF1 mRNA Levels in AGS-EBV Cell Line

AGS-EBV cell line was seeded in a 100 mm dish at 1×10⁶ cells/well and then transfected with 30 nM LNA control and 30 nM LNA-miR-BART20-5pi, respectively, using Lipofectamine 2000.

After 24 hours, the AGS-EBV cell line was treated with 5 nM TPA for 48 hours. After isolation of RNAs from both groups, cDNA was synthesized using a Mir-X™ First-Strand Synthesis kit, and then the BRLF1 and BZLF1 mRNA levels were measured by quantitative real-time PCR.

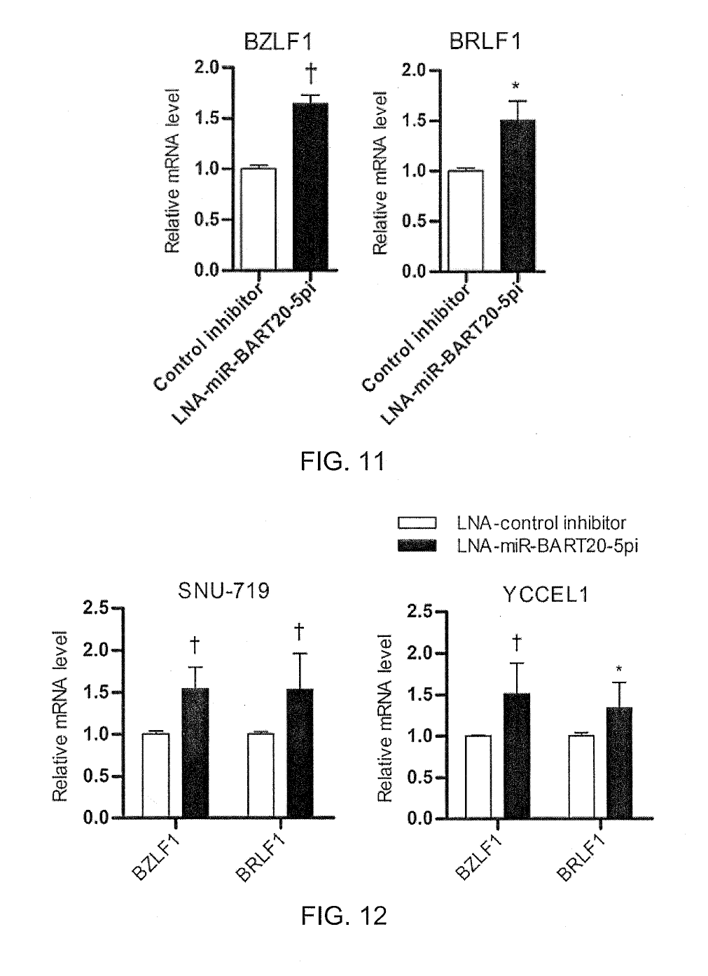

The results are shown in FIG. 11. It can be seen from FIG. 11 that the BRLF1 and BZLF1 mRNA levels were increased by more than 150% in the group treated with LNA-miR-BART20-5pi compared to the control.

(2) Determination of Increase in BRLF1 and BZLF1 mRNA Levels in SNU-719 Cell Line and YCCEL1 Cell Line

SNU-719 cell line and YCCEL1 cell line, EBV-positive cell lines isolated from Korean EBV-positive gastric cancer tissue, were prepared. Cells were seeded in a 6-well plate at 5×10⁴ cells/well, cultured for 24 hours, transfected with 50 nM LNA-miR-BART20-5pi and 50 nM LNA-control, respectively, and then cultured for 24 hours. Then, the cells were treated with TPA in the same manner as the above (1), and the expression of BRLF1 and BZLF1 mRNAs was measured by quantitative real-time PCR.

The results are shown in FIG. 12. It can be seen from FIG. 12 that the BRLF1 and BZLF1 mRNA levels were increased by more than about 150% in the group treated with LNA-miR-BART20-5pi compared to the control.

6-2. Determination of Increase in Expression of BRLF1, BZLF1, BMRF1, BALF5, and BHRF1 Proteins by Western Blot

AGS-EBV cell line was seeded in a 100 mm dish at 1×10⁶ cells/well and then transfected with 30 nM LNA control and 30 nM LNA-miR-BART20-5pi, respectively, using Lipofectamine 2000.

After 24 hours, the AGS-EBV cell line was divided into two groups, and only one group was treated with 5 nM TPA for 48 hours. After isolation of proteins from both groups, the proteins were subjected to electrophoresis on 8% SDS gels and transferred to a PVDF membrane, thus determining the expression level of lytic genes, BRLF1, BZLF1, and BALF5 proteins.

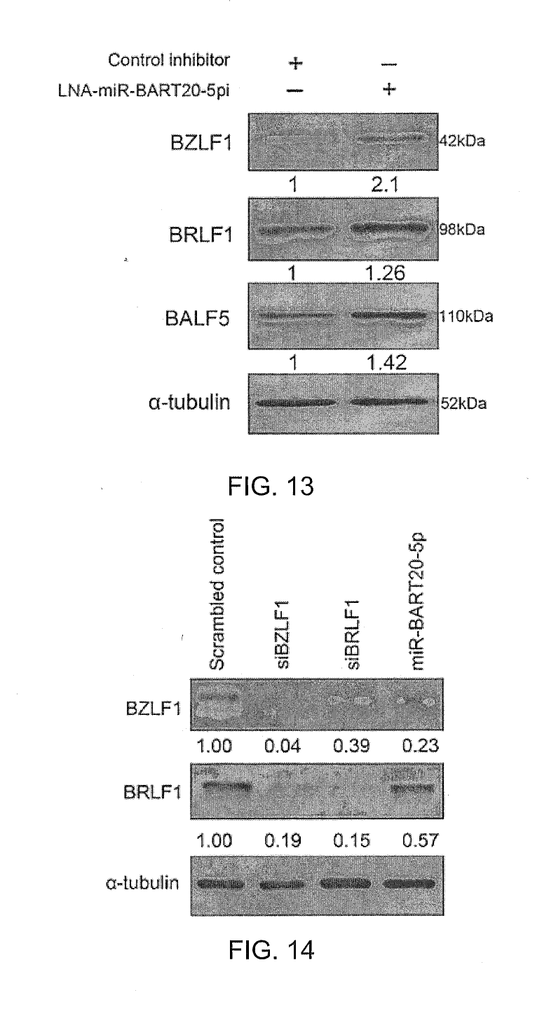

The Western blot results are shown in FIG. 13. It can be seen from FIG. 13 that the expression of BRLF1, BZLF1, and BALF5 proteins was increased in the group treated with LNA-miR-BART20-5pi compared to the control.

It can be seen from the results of Examples 6-1 and 6-2 that the LNA-miR-BART20-5pi induces the expression of lytic genes, BRLF1, BZLF1 and BALF5, thus inducing the lytic cycle of EBV.

### Example 7

### Comparison of Effect of miR-BART20-5p Mimic and that of siBRLF1 and siBZLF1

7-1. Comparison of Effect of miR-BART20-5p Mimic and that of siBRLF1 and siBZLF1 on Expression of BRLF1 and BZLF1

siRNAs that directly bind to BRLF1 and BZLF1 of EBV to inhibit the expression of BRLF1 and BZLF1 were synthesized and named siBRLF1 and siBZLF1, respectively.

The effects of siBRLF1, siBZLF1 and miR-BART20-5p mimic on the expression level of BRLF1 and BZLF1 proteins were determined by Western blot analysis in the same manner as in Example 6.

The results are shown in FIG. 14. It can be seen from FIG. 14 that siBRLF1 and siBZLF1 significantly reduced the expression level of BRLF1 and BZLF1 proteins, which was similar to that of the miR-BART20-5p mimic.

7-2. Comparison of Effect of miR-BART20-5p Mimic and that of siBRLF1 and siBZLF1 on Production of Viral Particles

AGS-EBV cell line was seeded in a 100 mm dish at 1×10⁶ cells/well and then transfected with either 30 nM siBRLF1 and siBZLF1 or 30 nM miR-BART20-5p mimic, respectively, using Lipofectamine 2000.

After 24 hours, the AGS-EBV cell line was divided into two groups, and only one group was treated with 5 nM TPA for 48 hours. After removing cells, the supernatant was filtered through a 0.45 μm filter, and then viral particle pellets were harvested using a centrifuge. After isolation of DNA from both groups, the amount of EBV genome was measured by PCR.

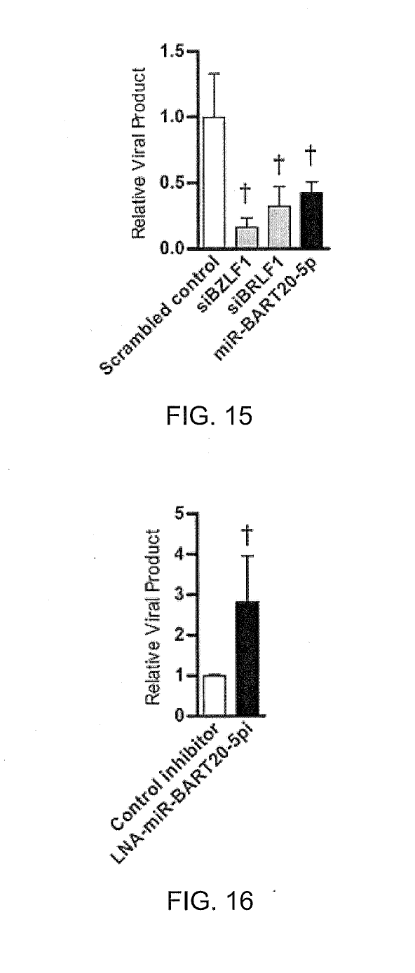

The results are shown in FIG. 15. It can be seen from FIG. 15 that miR-BART20-5p inhibited the production of progeny viruses at a level similar to that of siBRLF1 and siBZLF1.

### Example 8

### Determination of Effect of LNA-miR-BART20-5pi on Production of Viral Particles

AGS-EBV cell line was seeded in a 100 mm dish at 1×10⁶ cells/well and then transfected with either 30 nM control or 30 nM LNA-miR-BART20-5pi, respectively, using Lipofectamine 2000. Viral pellets were harvested in the same manner as in Example 7-2, followed by DNA isolation, and then the amount of EBV genome was measured by PCR to determine the production of progeny viruses.

The results are shown in FIG. 16. It can be seen from FIG. 16 that the production of progeny viruses was increased by LNA-miR-BART20-5pi by more than 2.5 times compared to the control.

What is claimed is:

1. A method for treating Epstein-Barr virus infection of a subject, comprising administering miR-BART20-5p inhibitor represented by SEQ ID NO: 1, SEQ ID NO: 2, or SEQ ID NO: 3 to a subject in need thereof.

2. The method of claim 1, wherein the Epstein-Barr virus infection is selected from the group consisting of granular fever, hairy leukoplakia, polymyositis, dermatomyositis, systemic lupus erythematosus, rheumatic arthritis, and cancer.

3. The method of claim 2, wherein the cancer is selected from the group consisting of Epstein-Barr virus-positive Burkitt's lymphoma, Hodgkin's lymphoma, nasopharyngeal cancer, nasal natural killer/T-cell lymphoma, and gastric cancer.

4. A method for inducing Epstein-Barr virus lytic cycle in a subject, comprising administering miR-BART20-5p inhibitor represented by SEQ ID NO: 1, SEQ ID NO: 2, or SEQ ID NO: 3 to a subject in need thereof.

5. A method for inhibiting the induction of Epstein-Barr virus lytic cycle ex vivo or in vitro, the method comprising the step of inhibiting the induction of Epstein-Barr virus lytic cycle by treating Epstein-Barr virus-positive cells with miR-BART20-5p represented by SEQ ID NO: 4 or a miR-BART20-5p analogue represented by SEQ ID NO: 5 or SEQ ID NO: 6.

6. The method of claim 6, wherein the inhibition of the induction of Epstein-Barr virus lytic cycle is achieved by inhibiting the expression of BRLF1 and BZLF1 of Epstein-Barr virus.

7. A method for screening a therapeutic agent for treating Epstein-Barr virus infection, the method comprising:
1) treating Epstein-Barr virus-positive cells with miR-BART20-5p represented by SEQ ID NO: 4 or a miR-BART20-5p analogue represented by SEQ ID NO: 5 or SEQ ID NO:6;
2) treating the cells with a candidate therapeutic agent; and
3) determining whether the induction of lytic cycle is induced in the cells.

8. The method of claim 7, wherein step 3) is carried out by determining whether the level of gene expression of Epstein-Barr virus is increased compared to that of latent Epstein-Barr virus positive cell.

9. The method of claim 8, wherein step 3) is carried out by determining whether BRLF1 and BZLF1 of Epstein-Barr virus are expressed in the cells.

10. A method for screening a therapeutic agent for treating Epstein-Barr virus infection, the method comprising the steps of:
1) treating cells that express BRLF1 3′UTR or BZLF1 3′UTR of Epstein-Barr virus with miR-BART20-5p represented by SEQ ID NO: 4 or a miR-BART20-5p analogue represented by SEQ ID NO: 5 or SEQ ID NO: 6;
2) treating the cells with a candidate therapeutic agent; and
3) determining whether BRLF1 3′UTR or BZLF1 3′UTR is expressed in the cells.


SEQUENCE LISTING (23 sequences)

SEQ ID NO: 1 - RNA, 21 nt, Artificial Sequence
  miR-BART20-5p inhibitor
nnnnnnnnnnnnngccugcun

SEQ ID NO: 2 - RNA, 21 nt, Artificial Sequence
  miR-BART20-5p inhibitor
nnanugaanncangccugcun

SEQ ID NO: 3 - RNA, 21 nt, Artificial Sequence
  LNA-miR-BART-20-5pi
ggaaugaagacaugccugcua

SEQ ID NO: 4 - RNA, 21 nt, Artificial Sequence
  miR-BART20-5p
uagcaggcaugucuucauucc

SEQ ID NO: 5 - RNA, 21 nt, Artificial Sequence
  miR-BART20-5p analogue
nagcaggcnugnnuucanunn

SEQ ID NO: 6 - RNA, 21 nt, Artificial Sequence
  miR-BART20-5p analogue
nagcaggcnnnnnnnnnnnnn

SEQ ID NO: 7 - RNA, 1032 nt, Epstein-Barr Virus
  BRLF1 3′UTR
acacuucugaaaacugccuccuccucuuuuagaaacuaugcaugagccacaggcauugcuaauguaccucauagacacaccuaaauuuagcacgucccaaaccaugacaucacagaggaggcuggugccuuggcuuuaaaggggagauguuagacagguaacucacuaaacauugcaccuugccggccaccuuugcuaucuuugcugaagaugauggacccaaacucgacuucugaagauguaaaauuuacaccugacccauaccaggugccuuuuguacaagcuuuugaccaagcuaccagagucuaucaggaccugggagggccaucgcaagcuccuuugccuugugugcuguggccggugcugccagagccucugccacaaggccagcuaacugccuaucauguuucaaccgcuccgacugggucgugguuuucugccccucagccugcuccugagaaugcuuaucaagcuuaugcagcaccucagcuguucccagucuccgacauaacccagaaucaacagacuaaccaagccgggggagaagcaccucaaccuggagacaauucuacuguucaaacagcagcagcagugguguuugcuugccccggggcuaaccaaggacaacagcuagcagacauugguguuccacagccugcaccaguggcugccccggcacgacgcacacggaaaccacaacagccagaaucgcuggaggaaugcgauucugaacuagaaauaaagcgauacaagaaucggguggcuuccagaaaaugccgggccaaguuuaagcaacugcugcagcacuaccgugagguggcugcugccaaaucaucugaaaaugacaggcugcgccuccuguugaagcagaugugcccaagccuggauguugacuccauuaucccccggacaccagauguuuuacacgaggaucucuuaaauuucuaacucccguuauugaaaccacgccugcuucacgccucguuuacuaauggaauauuaauaaauaucuuccugucggcucucuuugaa

SEQ ID NO: 8 - RNA, 84 nt, Epstein-Barr Virus
  BZLF1 3′UTR
cucccguuauugaaaccacgccugcuucacgccucguuuacuaauggaauauuaauaaauaucuuccugucggcucucuuugaa

SEQ ID NO: 9 - DNA, 29 bp, Artificial Sequence
  BRLF1 3′UTR forward primer
tcgactcgaggagccacaggcattgctaa

SEQ ID NO: 10 - DNA, 32 bp, Artificial Sequence
  BRLF1 3′UTR reverse primer
ggccgcggccgccaaagagagccgacaggaag

SEQ ID NO: 11 - DNA, 36 bp, Artificial Sequence
  BZLF1 3′UTR forward primer
tcgactcgagcgaggatctcttaaatttctaactcc

SEQ ID NO: 12 - DNA, 32 bp, Artificial Sequence; identical to SEQ ID NO: 10
  BZLF1 3′UTR reverse primer

SEQ ID NO: 13 - RNA, 25 nt, Artificial Sequence
  miR-BART20-5p scrambled control
ccucgugccguuccaucagguaguu

SEQ ID NO: 14 - RNA, 21 nt, Artificial Sequence
  miR-BART20-5pm
uaggucgcaugucuucauucc

SEQ ID NO: 15 - RNA, 84 nt, Artificial Sequence
  BZLF1m1
cucccguuauugaaaccacggcucgaucacgccucguuuacuaauggaauauuaauaaauaucuuccugucggcucucuuugaa

SEQ ID NO: 16 - RNA, 1032 nt, Artificial Sequence
  BRLF1m1
acacuucugaaaacugccuccuccucuuuuagaaacuaugcaugagccacaggcauugcuaauguaccucauagacacaccuaaauuuagcacgucccaaaccaugacaucacagaggaggcuggugccuuggcuuuaaaggggagauguuagacagguaacucacuaaacauugcaccuugccggccaccuuugcuaucuuugcugaagaugauggacccaaacucgacuucugaagauguaaaauuuacaccugacccauaccaggugccuuuuguacaagcuuuugaccaagcuaccagagucuaucaggaccugggagggccaucgcaagcuccuuugccuugugugcuguggccggugcugccagagccucugccacaaggccagcuaacugccuaucauguuucaaccgcuccgacugggucgugguuuucugccccucagccugcuccugagaaugcuuaucaagcuuaugcagcaccucagcuguucccagucuccgacauaacccagaaucaacagacuaaccaagccgggggagaagcaccucaaccuggagacaauucuacuguucaaacagcagcagcagugguguuugcuugccccggggcuaaccaaggacaacagcuagcagacauugguguuccacagccugcaccaguggcugccccggcacgacgcacacggaaaccacaacagccagaaucgcuggaggaaugcgauucugaacuagaaauaaagcgauacaagaaucggguggcuuccagaaaaugccgggccaaguuuaagcaacugcugcagcacuaccgugagguggcugcugccaaaucaucugaaaaugacaggcugcgccuccuguugaagcagaugugcccaagccuggauguugacuccauuaucccccggacaccagauguuuuacacgaggaucucuuaaauuucuaacucccguuauugaaaccacggcucgaucacgccucguuuacuaauggaauauuaauaaauaucuuccugucggcucucuuugaa

SEQ ID NO: 17 - RNA, 1032 nt, Artificial Sequence
  BRLF1m2
acacuucugaaaacugccuccuccucuuuuagaaacuaugcaugagccacaggcauugcuaauguaccucauagacacaccuaaauuuagcacgucccaaaccaugacaucacagaggaggcuggugccuuggcuuuaaaggggagauguuagacagguaacucacuaaacauugcaccuugccggccaccuuugcuaucuuugcugaagaugauggacccaaacucgacuucugaagauguaaaauuuacaccugacccauaccaggugccuuuuguacaagcuuuugaccaagcuaccagagucuaucaggaccugggagggccaucgcaagcuccuuugccuugugugcuguggccggugcugccagagccucugccacaaggccagcuaacugccuaucauguuucaaccgcuccgacugggucgugguuuucugccccucaggcucgaccugagaaugcuuaucaagcuuaugcagcaccucagcuguucccagucuccgacauaacccagaaucaacagacuaaccaagccgggggagaagcaccucaaccuggagacaauucuacuguucaaacagcagcagcagugguguuugcuugccccggggcuaaccaaggacaacagcuagcagacauugguguuccacagccugcaccaguggcugccccggcacgacgcacacggaaaccacaacagccagaaucgcuggaggaaugcgauucugaacuagaaauaaagcgauacaagaaucggguggcuuccagaaaaugccgggccaaguuuaagcaacugcugcagcacuaccgugagguggcugcugccaaaucaucugaaaaugacaggcugcgccuccuguugaagcagaugugcccaagccuggauguugacuccauuaucccccggacaccagauguuuuacacgaggaucucuuaaauuucuaacucccguuauugaaaccacgccugcuucacgccucguuuacuaauggaauauuaauaaauaucuuccugucggcucucuuugaa

SEQ ID NO: 18 - RNA, 1032 nt, Artificial Sequence
  BRLF1m1m2
acacuucugaaaacugccuccuccucuuuuagaaacuaugcaugagccacaggcauugcuaauguaccucauagacacaccuaaauuuagcacgucccaaaccaugacaucacagaggaggcuggugccuuggcuuuaaaggggagauguuagacagguaacucacuaaacauugcaccuugccggccaccuuugcuaucuuugcugaagaugauggacccaaacucgacuucugaagauguaaaauuuacaccugacccauaccaggugccuuuuguacaagcuuuugaccaagcuaccagagucuaucaggaccugggagggccaucgcaagcuccuuugccuugugugcuguggccggugcugccagagccucugccacaaggccagcuaacugccuaucauguuucaaccgcuccgacugggucgugguuuucugccccucaggcucgaccugagaaugcuuaucaagcuuaugcagcaccucagcuguucccagucuccgacauaacccagaaucaacagacuaaccaagccgggggagaagcaccucaaccuggagacaauucuacuguucaaacagcagcagcagugguguuugcuugccccggggcuaaccaaggacaacagcuagcagacauugguguuccacagccugcaccaguggcugccccggcacgacgcacacggaaaccacaacagccagaaucgcuggaggaaugcgauucugaacuagaaauaaagcgauacaagaaucggguggcuuccagaaaaugccgggccaaguuuaagcaacugcugcagcacuaccgugagguggcugcugccaaaucaucugaaaaugacaggcugcgccuccuguugaagcagaugugcccaagccuggauguugacuccauuaucccccggacaccagauguuuuacacgaggaucucuuaaauuucuaacucccguuauugaaaccacggcucgaucacgccucguuuacuaauggaauauuaauaaauaucuuccugucggcucucuuugaa

SEQ ID NO: 19 - DNA, 30 bp, Artificial Sequence
  BRLF1m2 forward primer
ctgagaatgcttatcaagcttatgcagcac

SEQ ID NO: 20 - DNA, 27 bp, Artificial Sequence
  BRLF1m2 reverse primer
gtcgagcctgaggggcaggaaaccacg

SEQ ID NO: 21 - DNA, 35 bp, Artificial Sequence
  BRLF1m1 forward primer
cacgcctcgtttactaatggaatattaataaatat

SEQ ID NO: 22 - DNA, 23 bp, Artificial Sequence
  BRLF1m1 reverse primer
atcgagccgtggtttcaataacg

SEQ ID NO: 23 - DNA, 19 bp, Artificial Sequence
  LNA control
taacacgtctatacgccca